(19) United States
(12) Patent Application Publication
(10) Pub. No.: US 2024/0358854 A1
(43) Pub. Date: Oct. 31, 2024

(54) METHOD FOR GENE EDITING USING GENE THERAPY

(71) Applicant: CARDIAI TECHNOLOGIES, Calgary (CA)

(72) Inventors: Minal Borkar Tripathi, Calgary (CA); Sidharth Singh Bhinder, Calgary (CA); Anmol Singh Kapoor, Calgary (CA); Amrita Kaur, Calgary (CA)

(21) Appl. No.: 18/647,036

(22) Filed: Apr. 26, 2024

Related U.S. Application Data
(60) Provisional application No. 63/498,638, filed on Apr. 27, 2023.

(51) Int. Cl. A61K 48/00; C12N 9/22; C12N 15/11; C12N 15/85

(52) U.S. Cl. CPC A61K 48/005; C12N 9/22; C12N 15/111; C12N 15/85; C12N 2310/20

(57) ABSTRACT

The present disclosure relates to a method (100, 200) for gene editing of a cell or tissue. The method (100,200) includes introducing a gene editing agent into the cell or tissue using a gene delivery vector. The method (100,200) also includes modifying the gene delivery vector with a targeting agent to direct the gene editing agent to the desired cell or tissue. The method (100,200) also includes attuning the gene delivery agent to modify or replace the mutated gene. The method (100,200) also includes introducing other genetic modifications through the genetic agent. The method (100,200) also includes modifying the gene delivery vector with agents to enhance the stability or activity of the gene editing agent.

### CROSS-REFERENCE TO RELATED APPLICATIONS

This application claims the benefit of U.S. Provisional Application No. 63/498,638 titled “A METHOD FOR GENE EDITING USING GENE THERAPY” filed by the applicant on Apr. 27, 2023, which is incorporated herein by reference in its entirety.

### FIELD OF THE INVENTION

Embodiments of the present invention relate to a method for gene manipulation and more specifically to methods for gene editing using gene therapy.

### DESCRIPTION OF THE RELATED ART

Genomics is the study of the structure, function, and evolution of genomes. It is a field at the intersection of genetics and molecular biology, focuses on the structure, function, evolution, mapping, and editing of genomes, the complete set of an organism's deoxyribonucleic acid (DNA), including all of its genes. It involves the sequencing and analysis of deoxyribonucleic acid (DNA) and ribonucleic acid (RNA) molecules, as well as the identification of the genes contained in these molecules. The understanding of the genetic code and the ability to identify and analyze particular genes are essential for the development of new treatments for diseases.

The genes hold the blueprint for an organism's development, functioning, and inheritance, making genomics a crucial discipline in understanding life at its most fundamental level. Since its inception, the primary goal of genomics is decoding the genetic code underlying various traits and diseases. If any gene is mutated, it can lead to development of a disease.

In particularly, many of the both rare and common diseases are caused by genetic mutations. So, identifying and correcting the specific genes involved in development of such conditions is crucial for accurate diagnosis, prognosis, and treatment.

In addition to this, gene editing is often used to treat genetic diseases caused by mutations in genes, but it can also be used to modify certain traits or characteristics of an organism. In particular, gene editing has revolutionized the field of genetic research, allowing scientists to study the function of genes and their role in disease. Furthermore, it has also shown promise in the development of new treatments for genetic diseases, including inherited disorders such as cystic fibrosis, sickle cell anemia, and Huntington's disease.

By modifying the underlying genetic cause of these diseases, gene editing has the potential to provide long-lasting cures, rather than simply managing the symptoms. As such, there is a need for careful regulation of gene editing technology to ensure that it is used responsibly and safely.

It also has important implications for agriculture, where gene editing can be used to produce crops that are more resistant to pests or drought. It can also be used to produce livestock that are resistant to diseases or that produce more meat or milk.

Although, gene editing technologies have revolutionized biological research and hold immense promise for applications in medicine, agriculture, and beyond. However, they also come with many drawbacks and limitations: While existing gene therapies are highly efficient at targeting specific sequences within the genome, its precision is not absolute. Small variations in the target sequence or limitations in the delivery mechanism can result in incomplete or inaccurate editing, reducing the reliability of the technique.

Yet another significant limitation is associated with delivery mechanism for gene editing. Getting gene-editing tools into the target cells or tissues can be challenging, particularly in complex organisms like humans. Current delivery methods often have low efficiency, limiting the practical application of gene editing in certain contexts.

Furthermore, many traits and diseases are influenced by multiple genes and environmental factors, making them challenging to edit using current techniques. Gene editing may be less effective or even impractical for addressing complex traits and polygenic disorders. Addressing these drawbacks and limitations require a thoughtful approach to balancing the potential benefits of gene editing with its ethical, social, and safety considerations.

Accordingly, to overcome the disadvantages of the prior art, there is an urgent need for a technical solution that overcomes the above-stated limitations in the prior arts. The present invention provides a method for gene editing using gene therapy.

### SUMMARY OF THE INVENTION

The present disclosure solves all the above major limitations of a method for gene editing using gene therapy. Further, the present disclosure ensures that the disclosed invention may fulfil following objectives.

An objective of the present disclosure is to provide an effective and reliable method for editing a particular gene.

Another objective of the present disclosure is to develop a less complex method for introducing a gene editing agent into the cell or tissue using a gene delivery vector.

Another objective of the present disclosure is to develop a modified gene delivery vector with a targeting agent to direct the gene editing agent to the desired cell or tissue.

Another objective of the present disclosure is to develop cost effective method for increasing the efficiency of gene delivery.

Another objective of the present disclosure is to allow for precise targeting of specific genes or genetic sequences within an organism's genome, minimizing off-target effects and unintended consequences.

Another objective of the present disclosure is to enhance the efficiency of gene editing processes to enable reliable and consistent modifications across a wide range of cell types and organisms.

Another objective of the present disclosure is to advance the understanding and manipulation of genetic material in a responsible manner.

Yet another objective of the present disclosure is to facilitate research and applications in fields such as medicine, agriculture, and biotechnology.

Yet another objective of the present disclosure is to create versatile gene editing method that can be adapted to various applications, including correcting genetic disorders, enhancing crop traits, studying gene function, and developing new therapies and treatments.

Yet another objective of the present disclosure is to ensure that gene editing method is safe for use in both laboratory and clinical settings, minimizing the risk of adverse effects on organisms and ecosystems.

Embodiments of the present invention relate to a method (100) for gene editing of a cell or tissue. The method includes introducing a gene editing agent into the cell or tissue using a gene delivery vector. The method also includes modifying the gene delivery vector with a targeting agent to direct the gene editing agent to the desired cell or tissue. The method also includes attuning the gene delivery agent to modify or replace the mutated gene. The method also includes introducing other genetic modifications through the genetic agent. The method also includes modifying the gene delivery vector with agents to enhance the stability or activity of the gene editing agent.

In accordance with an embodiment of the present invention, the gene editing agent is selected from the group consisting of clustered regularly interspaced short palindromic repeats (CRISPR)-Cas9 and Zinc Finger Nucleases.

In accordance with an embodiment of the present invention, the gene delivery agent is a viral vector or a non-viral vector.

In accordance with an embodiment of the present invention, the use of gene editing agent to modify or replace the mutated gene allow the cell or tissue to produce a functional protein product.

In accordance with an embodiment of the present invention, the genetic modifications include, gene knockouts or gene knockins.

In accordance with an embodiment of the present invention, the modified gene is a naturally occurring gene or a synthetic gene.

In accordance with an embodiment of the present invention, the accommodation of both naturally occurring genes and synthetic genes offers flexibility in the types of genetic modifications introduced.

In accordance with an embodiment of the present invention, the accommodation of both naturally occurring genes and synthetic genes enables development of tailored approaches for different genetic disorders and therapeutic needs.

In accordance with an embodiment of the present invention, the modification of gene delivery vectors with transfection agents increases the efficiency of gene delivery, ensuring a higher proportion of target cells receive the gene editing agent and resulting in more effective therapeutic outcomes.

In accordance with an embodiment of the present invention, the use of gene delivery vectors modified with targeting agents enables precise delivery of gene editing agents to specific cells or tissues, enhancing the specificity of gene editing therapies.

In accordance with an embodiment of the present invention, the incorporation of stability enhancers into gene delivery vectors helps protect the gene editing agents from degradation, ensuring their functionality and activity are maintained throughout the editing process.

### BRIEF DESCRIPTION OF THE DRAWINGS

So that the manner in which the above-recited features of the present invention is understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

The invention herein will be better understood from the following description with reference to the drawings, in which:

FIG. 1 is a flowchart illustrating a method for gene editing of a cell or tissue, in accordance with an embodiment of the present invention.

The method for editing gene using gene therapy is illustrated in the accompanying drawings, which like reference letters indicate corresponding parts in the various figures. It should be noted that the accompanying FIGURE is intended to present illustrations of exemplary embodiments of the present disclosure. This FIGURE is not intended to limit the scope of the present disclosure. It should also be noted that the accompanying FIGURE is not necessarily drawn to scale.

### DETAILED DESCRIPTION OF THE INVENTION

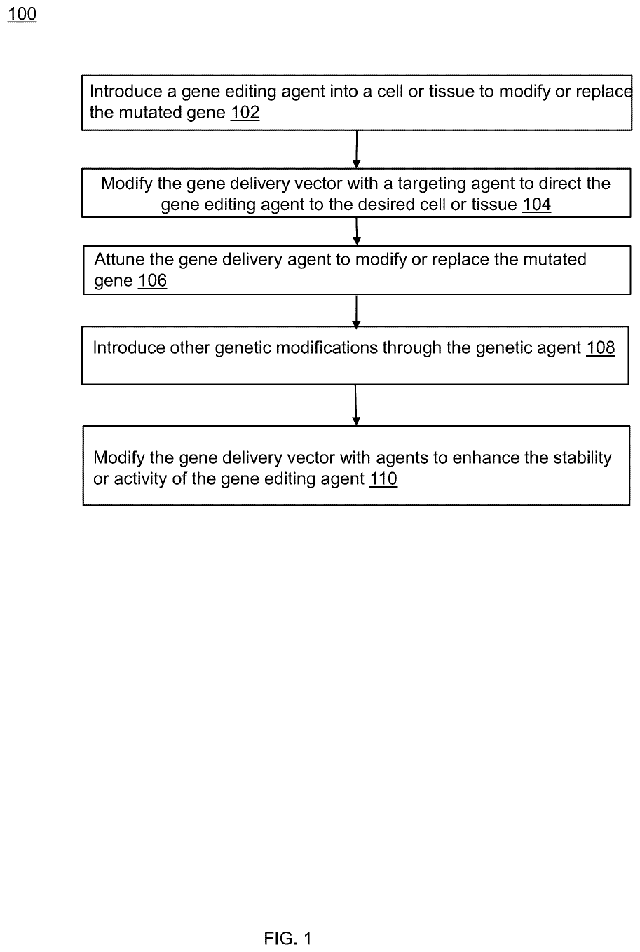

The principles of the present invention and their advantages are best understood by referring to FIG. 1. In the following detailed description, numerous specific details are set forth in order to provide a thorough understanding of the embodiment of the invention as illustrative or exemplary embodiments of the invention, specific embodiments in which the invention may be practiced are described in sufficient detail to enable those skilled in the art to practice the disclosed embodiments. However, it will be obvious to a person skilled in the art that the embodiments of the invention may be practiced with or without these specific details. In other instances, well-known methods, procedures and components have not been described in detail so as not to unnecessarily obscure aspects of the embodiments of the invention.

The following detailed description is, therefore, not to be taken in a limiting sense, and the scope of the present invention is defined by the appended claims and equivalents thereof. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. References within the specification to “one embodiment,” “an embodiment,” “embodiments,” or “one or more embodiments” are intended to indicate that a particular feature, structure, or characteristic described in connection with the embodiment is included in at least one embodiment of the present invention.

Although the terms first, second, etc. may be used herein to describe various elements, these elements should not be limited by these terms. These terms are generally only used to distinguish one element from another and do not denote any order, ranking, quantity, or importance, but rather are used to distinguish one element from another. Further, the terms “a” and “an” herein do not denote a limitation of quantity, but rather denote the presence of at least one of the referenced items.

The conditional language used herein, such as, among others, “can,” “may,” “might,” “may,” “e.g.,” and the like, unless specifically stated otherwise, or otherwise understood within the context as used, is generally intended to convey that certain embodiments include, while other embodiments do not include, certain features, elements and/or steps.

Disjunctive language such as the phrase “at least one of X, Y, Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

The following brief definition of terms shall apply throughout the present invention:

FIG. 1 is a flowchart illustrating a method 100 for gene editing of a cell or tissue, in accordance with an embodiment of the present invention. The method 100 may comprise following steps.

At 102, introducing a gene editing agent into the cell or tissue using a gene delivery vector.

At 104, modifying the gene delivery vector with a targeting agent to direct the gene editing agent to the desired cell or tissue.

At 106, attuning the gene delivery agent to modify or replace the mutated gene. At 108, introducing other genetic modifications through the genetic agent.

At 110, modifying the gene delivery vector with agents to enhance the stability or activity of the gene editing agent.

The gene editing agent may be selected from the group consisting of clustered regularly interspaced short palindromic repeats (CRISPR)-Cas9 and Zinc Finger Nucleases.

The gene delivery agent may be a viral vector or a non-viral vector.

The use of gene editing agent may modify or replace the mutated gene allow the cell or tissue to produce a functional protein product.

The genetic modifications may include, gene knockouts or gene knockins.

The modified gene may be a naturally occurring gene or a synthetic gene.

The accommodation of both naturally occurring genes and synthetic genes may offer flexibility in the types of genetic modifications introduced, and enable development of tailored approaches for different genetic disorders and therapeutic needs.

The modification of gene delivery vectors with transfection agents may increase the efficiency of gene delivery, ensuring a higher proportion of target cells receive the gene editing agent and resulting in more effective therapeutic outcomes.

The use of gene delivery vectors modified with targeting agents may enable precise delivery of gene editing agents to specific cells or tissues, enhancing the specificity of gene editing therapies.

The incorporation of stability enhancers into gene delivery vectors may help protect the gene editing agents from degradation, ensuring their functionality and activity are maintained throughout the editing process.

In an embodiment of the present disclosure, choice of the gene delivery vector is based on factors such as the type of cells or tissues being targeted and the size of the clustered regularly interspaced short palindromic repeats (CRISPR)-Cas9 and Zinc Finger Nucleases components, and efficiency of delivery. In some embodiments, the viral vectors may include lentiviruses, adenoviruses, or adeno-associated viruses (AAVs), used due to their high efficiency in delivering genetic material into cells.

In an embodiment of the present disclosure, the viral vectors may be engineered to be replication-deficient and safe for use in laboratory and clinical settings. In some embodiments, the viral vector may be engineered to carry the gene editing agent within its genome. When introduced into the target cells, the viral vector infects the cells and delivers the edited component, allowing the gene editing to perform its function.

In an embodiment of the present disclosure, the non-viral vectors offer advantages such as reduced immunogenicity, simpler manufacturing, and potentially lower toxicity compared to viral vectors. In some embodiments, the non-viral vectors may include liposomes, nanoparticles, and electroporation. In some embodiments, the non-viral vectors are typically introduced into cells via transfection, where the vector is combined with transfection reagents and added directly to the cell culture. Alternatively, physical methods like electroporation can be used to create temporary pores in the cell membrane, allowing the vector to enter the cell.

In some embodiments, to optimize delivery conditions, parameters like vector concentration, incubation time, and cell density are altered to maximize the efficiency of gene delivery while minimizing cytotoxicity and off-target effects. In some embodiments, for assess the efficiency of gene editing within the target cells using techniques such as PCR, sequencing, or functional assays to confirm successful introduction and activity of the gene editing agent.

In an embodiment of the present disclosure, the strategies for introducing the gene editing agent into tissue or cell may include direct injection into tissues or organs, systemic administration via intravenous or intramuscular injection, or targeted delivery using nanoparticles or other carriers.

In an embodiment of the present disclosure, the targeting agent may recognize and bind to receptors or markers expressed on the surface of the target cells or tissues. In some embodiments, the targeting agent may be a ligand, antibody, aptamer, peptide, or other molecule with high affinity and specificity for the target. In some embodiment, the targeting agent may conjugate or fuse with the gene delivery vector to create a modified vector with targeting capability. In some embodiments, chemical conjugation, genetic fusion, or other bioconjugation methods are used for modifying the gene delivery vector with a targeting agent.

In some embodiments, modification of gene delivery vector allows for efficient recognition and binding to the target cells or tissues. This may involve incorporating specific binding motifs or modifying surface proteins to display the targeting moiety. In some embodiments, cell culture models or ex vivo tissue samples may be used to validate the effectiveness of the targeting agent in the gene delivery to the desired cells or tissues while minimizing off-target interactions.

In some embodiments, the attuning of the gene delivery agent may be related to optimizing targeting specificity, delivery efficiency, safety profile, and/or such. In some embodiments, the attuning of the gene delivery agent may be performed by incorporating targeting ligands or peptides that recognize and bind to specific receptors or markers on the surface of the target cells or tissues. In some embodiments, the attuning of the gene delivery agent may involve modifying the vector's size, charge, or surface properties to enhance cellular uptake and intracellular delivery.

In an embodiment of the present disclosure, introducing additional genetic modifications through the gene delivery agent involves expanding its capabilities beyond delivering gene editing agent. It incorporates or deliver other genetic elements such as transgenes, RNA interference (RNAi) constructs, or regulatory elements. In some embodiments, the gene delivery vector may introduce desired traits or functions into target cells or tissues. For example, it may involve delivering therapeutic genes for gene therapy applications, reporter genes for tracking cells in vivo, and/or such.

In some embodiments, the gene delivery agent may deliver small interfering RNA (siRNA) or short hairpin RNA (shRNA) constructs to induce RNA interference and silence specific target genes to downregulate the expression of disease-causing genes or study gene function. In some embodiments, the gene delivery vector may be used to control the expression levels or patterns of delivered genes. In some embodiments, the gene delivery agent may also allow for simultaneous modification of multiple genes within the same cell, modulate gene expression or epigenetic states within target cells or tissues, and incorporate inducible or conditional gene expression.

In some embodiments, modification of the gene delivery vector with agents may be performed by incorporating stabilizing agents into the gene delivery vector to protect from degradation, conjugate cell-penetrating peptides to enhance cellular uptake and intracellular delivery, incorporate endosomal escape agents to facilitate the release of the gene editing agent from endosomes into the cytoplasm following cellular uptake, include nuclear localization signals (to promote the nuclear import of the gene editing agent after cellular uptake, and more.

In a best mode of operation, the method 100 include delivery of gene editing agents to the target cells or tissues to modify or replace mutated genes using a gene delivery vector, which can be a viral or non-viral vector, that can transport the gene editing agent to the targeted cells or tissues. The gene delivery vector may be modified with a targeting agent to direct the gene editing agent to the desired cell or tissue. The targeting agent can be a protein, antibody, or aptamer that binds to a specific receptor or molecule on the surface of the target cell. This ensures that the gene editing agent may be delivered specifically to the intended cells, minimizing off-target effects. The gene editing agent itself may be any of the commonly used gene editing tools, such as CRISPR-Cas9, Zinc Finger Nucleases, or TALENs. Once delivered to the target cell, the gene editing agent may modify or replace the mutated gene, allowing the cell or tissue to produce a functional protein product.

The key advantage of the method 100 that it may expands the range of genetic modifications that can be introduced using gene editing. By incorporating gene editing using gene therapy, it can find utility in a wide range of applications, including functional genomics, regenerative medicine, and biotechnology. Mainly, the method 100 hold promise for addressing complex diseases, elucidating gene function, and engineering cells with desired traits for various biomedical and research purposes.

Another advantage of the method 100 is associated to its scalability and amenability to large-scale production for clinical or commercial applications. The method 100 is robust and ensure consistency and reproducibility of the delivery agent's performance. Another advantage relates to the attuning the gene delivery agent that can improve overall efficacy, safety, and suitability of the method 100.

In conclusion, the gene editing using gene therapy involves using tools like clustered regularly interspaced short palindromic repeats (CRISPR)-Cas9 to modify a patient's DNA to treat or prevent genetic disorders. This method 100 holds tremendous potential for addressing previously incurable diseases by correcting the underlying genetic mutations responsible for the condition. The method 100 typically involves delivering the gene editing components, such as the clustered regularly interspaced short palindromic repeats (CRISPR)-Cas9, into the target cells, where they can then make precise changes to the faulty genes.

The method 100 involves introducing a gene editing agent, such as clustered regularly interspaced short palindromic repeats (CRISPR)-Cas9 or Zinc Finger Nucleases, into a cell or tissue to modify or replace the mutated gene. The gene editing agent may be delivered to the cell or tissue utilizing a gene delivery vector. The delivery vector may be modified with a targeting agent to direct the gene editing agent to the desired cell or tissue such as a viral vector or a non-viral vector. Viral vectors, such as retroviruses, lentiviruses, adenoviruses, and adeno-associated viruses (AAVs), may be utilized due to their ability to infect cells and integrate the gene editing machinery into the cell's genome. Non-viral vectors, including plasmid DNA, liposomes, nanoparticles, and polymer-based delivery systems, may offer advantages such as reduced immunogenicity and the ability to carry larger genetic payloads. These vectors may be engineered with surface modifications to enhance targeting specificity, allowing them to deliver gene editing agents to specific cell types or tissues while minimizing interactions with non-target cells.

The gene editing agent may be used to modify or replace the mutated gene, thereby allowing the cell or tissue to produce a functional protein product. The modified gene may be a naturally occurring gene or a synthetic gene. The gene editing agent may also be utilized to introduce other genetic modifications, such as gene knockouts or gene knockins. The gene delivery vector may also be modified with other agents, such as transfection agents, to increase the efficiency of gene delivery. The gene delivery vector may also be modified with agents to enhance the stability of the gene editing agent. In addition to stability enhancements, gene delivery vectors may be modified with agents that enhance the activity of the gene editing agent.

For instance, small molecules or peptides may be included in the vector design to increase the efficiency of clustered regularly interspaced short palindromic repeats (CRISPR)-Cas9-mediated gene editing by promoting DNA cleavage or improving the binding affinity of the editing complex to its target sequence. These enhancers may improve the precision and efficacy of gene editing, leading to more accurate modifications of the target genes. Furthermore, the vector may be equipped with regulatory elements, such as inducible promoters or microRNA-responsive elements, to control the timing and level of gene editing activity. This level of control allows for fine-tuning the gene editing process, minimizing off-target effects, and enhancing the safety profile of the therapy.

In a case that no conflict occurs, the embodiments in the present disclosure and the features in the embodiments may be mutually combined. The foregoing descriptions are merely specific implementations of the present disclosure, but are not intended to limit the protection scope of the present disclosure. Any variation or replacement readily figured out by a person skilled in the art within the technical scope disclosed in the present disclosure shall fall within the protection scope of the present disclosure. Therefore, the protection scope of the present disclosure shall be subject to the protection scope of the claims.

The foregoing descriptions of specific embodiments of the present technology have been presented for purposes of illustration and description. They are not intended to be exhaustive or to limit the present technology to the precise forms disclosed, and obviously many modifications and variations are possible in light of the above teaching. The embodiments were chosen and described in order to best explain the principles of the present technology and its practical application, to thereby enable others skilled in the art to best utilize the present technology and various embodiments with various modifications as are suited to the particular use contemplated. It is understood that various omissions and substitutions of equivalents are contemplated as circumstance may suggest or render expedient, but such are intended to cover the application or implementation without departing from the spirit or scope of the claims of the present technology.

What is claimed for:

1. A method (100) for gene editing of a cell, the method (100) comprising:
introducing a gene editing agent into the cell using a gene delivery vector;
modifying the gene delivery vector with a targeting agent to direct the gene editing agent to the desired cell;
attuning the gene delivery agent to modify or replace the mutated gene;
introducing other genetic modifications through the genetic agent; and
modifying the gene delivery vector with agents to enhance the stability or activity of the gene editing agent.

2. The method of claim 1, wherein the gene editing agent is selected from the group consisting of clustered regularly interspaced short palindromic repeats (CRISPR)-Cas9 and zinc finger nucleases.

3. The method of claim 1, wherein the gene delivery agent is a viral vector or a non-viral vector.

4. The method of claim 1, wherein the use of gene editing agent to modify or replace the mutated gene allow the cell to produce a functional protein product.

5. The method of claim 1, wherein the genetic modifications include, gene knockouts or gene knockins.

6. The method of claim 1, wherein the modified gene is a naturally occurring gene or a synthetic gene.

7. The method of claim 6, wherein the accommodation of both naturally occurring genes and synthetic genes:
offers flexibility in the types of genetic modifications introduced; and
enables development of tailored approaches for different genetic disorders and therapeutic needs.

8. The method of claim 1, wherein the modification of gene delivery vectors with transfection agents increases the efficiency of gene delivery, ensuring a higher proportion of target cells receive the gene editing agent and resulting in more effective therapeutic outcomes.

9. The method of claim 1, wherein the use of gene delivery vectors modified with targeting agents enables precise delivery of gene editing agents to specific cells, enhancing the specificity of gene editing therapies.

10. The method of claim 1, wherein the incorporation of stability enhancers into gene delivery vectors helps protect the gene editing agents from degradation, ensuring their functionality and activity are maintained throughout the editing process.

11. A method (100) for gene editing of a tissue, the method (100) comprising:
introducing a gene editing agent into the tissue using a gene delivery vector;
modifying the gene delivery vector with a targeting agent to direct the gene editing agent to the desired tissue;
attuning the gene delivery agent to modify or replace the mutated gene;
introducing other genetic modifications through the genetic agent; and
modifying the gene delivery vector with agents to enhance the stability or activity of the gene editing agent.

12. The method of claim 1, wherein the gene editing agent is selected from the group consisting of clustered regularly interspaced short palindromic repeats (CRISPR)-Cas9 and zinc finger nucleases.

13. The method of claim 1, wherein the gene delivery agent is a viral vector or a non-viral vector.

14. The method of claim 1, wherein the use of gene editing agent to modify or replace the mutated gene allow the tissue to produce a functional protein product.

15. The method of claim 1, wherein the genetic modifications include, gene knockouts or gene knockins.

16. The method of claim 1, wherein the modified gene is a naturally occurring gene or a synthetic gene.

17. The method of claim 6, wherein the accommodation of both naturally occurring genes and synthetic genes:
offers flexibility in the types of genetic modifications introduced; and
enables development of tailored approaches for different genetic disorders and therapeutic needs.

18. The method of claim 1, wherein the modification of gene delivery vectors with transfection agents increases the efficiency of gene delivery, ensuring a higher proportion of target cells receive the gene editing agent and resulting in more effective therapeutic outcomes.

19. The method of claim 1, wherein the use of gene delivery vectors modified with targeting agents enables precise delivery of gene editing agents to specific tissues, enhancing the specificity of gene editing therapies.

20. The method of claim 1, wherein the incorporation of stability enhancers into gene delivery vectors helps protect the gene editing agents from degradation, ensuring their functionality and activity are maintained throughout the editing process.
